(19) United States
(12) Patent Application Publication
(10) Pub. No.: US 2025/0017734 A1
(43) Pub. Date: Jan. 16, 2025

(54) PIEZOELECTRIC NON-RETURN VALVE SHUT-OFF

(71) Applicant: Boston Scientific Scimed, Inc., Maple Grove, MN (US)

(72) Inventors: Richard Percy, Leamlara (IE); Michael Dunne, Castlecomer (IE); Daragh Nolan, Youghal (IE); Thomas Sinnott, Enniscorthy (IE); Liam Barry, Clonmel (IE); Orlaith Cotter, Clonmel (IE); Andrew Gibson, Limerick (IE)

(21) Appl. No.: 18/765,820

(22) Filed: Jul. 8, 2024

Related U.S. Application Data
(60) Provisional application No. 63/512,931, filed on Jul. 11, 2023.

(51) Int. Cl. A61F 2/26; A61F 2/48; F16K 7/14; F16K 31/00

(52) U.S. Cl. CPC A61F 2/26; A61F 2/484; F16K 7/14; F16K 31/005; A61F 2250/0013; A61F 2250/007

(57) ABSTRACT

An implantable fluid operated device includes a fluid control system to transfer fluid between a fluid reservoir and an inflatable member. The fluid control system includes at least one fluid control device including at least one pump and at least one valve, or at least one combination pump and valve device. A seal is provided in the at least one fluid control device. In a first mode, the seal provides a seal between a fluid passageway and a fluid chamber of the fluid control device, to close the fluid control device. In a second mode, the seal is disengaged to open the fluid control device and allow fluid to flow between the fluid passageway and the fluid chamber. A seal retention device maintains a position of the seal when the fluid control device is open and fluid is flowing through the fluid control device.

### CROSS-REFERENCE TO RELATED APPLICATION

This application claims priority to U.S. Provisional Patent Application No. 63/512,931, filed on Jul. 11, 2023, entitled “PIEZOELECTRIC NON-RETURN VALVE SHUT-OFF”, the disclosure of which is incorporated by reference herein in its entirety.

### TECHNICAL FIELD

This disclosure relates generally to bodily implants, and more specifically to bodily implants including a fluid control system having one or more pumps and/or valves including a piezoelectric actuator.

### BACKGROUND

Active implantable fluid operated inflatable devices can include one or more pumps that regulate a flow of fluid between different portions of the implantable device. One or more valves can be positioned within fluid passageways of the device to direct and control the flow of fluid to achieve inflation, deflation, pressurization, depressurization, activation, deactivation and the like of different fluid filled components of the device. In some implantable fluid operated devices, an implantable pumping device may be manually operated by the user to provide for the transfer of fluid between a reservoir and the fluid filled implant components of the device. In some situations, manual operation of the pumping device may make it difficult to achieve consistent inflation, deflation, pressurization, depressurization, activation, deactivation and the like of the fluid filled implant components. Inconsistent inflation, deflation, pressurization, depressurization, activation and/or deactivation of the fluid filled implant device(s) may adversely affect patient comfort, efficacy of the device, and the overall patient experience. Some implantable fluid operated devices include an electronic control system including an electronically controlled manifold providing for the transfer of fluid within the implantable fluid operated device. The use of the electronic control system may provide for more accurate actuation and control of the flow of fluid between components of the inflatable device, thus improving performance and efficacy of the device, as well as patient comfort and safety. Consistent inflation, deflation, pressurization, depressurization, activation, deactivation and the like of the fluid filled implant components may rely on accurate flow control through the pumps and/or valves within a manifold of the electronic control system. Setting and maintaining set positions of various components of the pumps and/or valves in the manifold may provide for consistent flow of fluid through the manifold, and for consistent, accurate control of the inflation, deflation, pressurization, depressurization, activation, deactivation and the like of the fluid filled components of the implantable fluid operated inflatable device.

### SUMMARY

In a general aspect, an implantable fluid operated inflatable device includes a housing; at least one fluid passageway within the housing; and a fluid control device positioned in the at least one fluid passageway, the fluid control device including a base plate; a diaphragm coupled to the base plate; a fluid chamber defined between the base plate and the diaphragm; an opening formed in the base plate, the opening connecting the fluid chamber to a fluid passageway of the fluid control device; a scal provided at the opening and configured to form a seal between the fluid passageway and the fluid chamber in a closed state of the fluid control device; and a seal retention mechanism engaged with the seal and configured to retain a position of the seal in an open state of the fluid control device.

In some implementations, the seal includes an O-ring positioned in a recess surrounding the opening formed in the base plate; and the seal retention mechanism includes a texturing layer formed on a surface of the recess; and a bonding layer between the texturing layer and the O-ring, the bonding layer coupling the O-ring in the recess via the texturing layer.

In some implementations, the seal includes an elastomer O-ring received in a recess surrounding the opening formed in the base plate; and the seal retention mechanism includes an undercut portion formed at an opening into the recess, wherein a dimension of the opening is less than a corresponding dimension of the elastomer O-ring such that the elastomer O-ring is retained in the recess by the undercut portion.

In some implementations, the seal is an elastomer O-ring, and wherein the seal retention mechanism includes a recess surrounding the opening formed in the base plate, with the elastomer O-ring received in the recess, wherein a dimension of an inner peripheral portion of the recess is greater than a corresponding dimension of the O-ring, such that the O-ring is retained in the recess by a compression force exerted by the O-ring on the inner peripheral portion of the recess, or, a dimension of an outer peripheral portion of the recess is less than a corresponding dimension of the O-ring, such that the O-ring is retained in the recess by a tensile force exerted by the O-ring on the outer peripheral portion of the recess.

In some implementations, the seal is insert-molded into the base plate, the seal including a body portion; a flange portion at a first end portion of the body portion; a sealing portion at a second end portion of the body portion; and an opening formed in the body portion, and the seal retention mechanism includes an undercut portion formed in a body portion of the base plate and configured to engage the flange portion of the seal; and molding material received through the opening in the body portion of the seal and integrally formed between a body portion and a central opening of the base plate.

In some implementations, the seal includes a body portion; a flange portion at a first end portion of the body portion; a scaling portion at a second end portion of the body portion, the scaling portion extending through a corresponding opening in the base plate; and an opening extending through the body portion, from the first end portion to the second end portion of the body portion, and the seal retention mechanism includes an undercut portion formed in a body portion of the base plate and configured to engage an upper portion of the flange portion of the seal; and a support plate coupled to a bottom portion of the base plate and configured to engage a bottom portion of the flange portion of the seal, such that the flange portion of the seal is retained between the base plate and the support plate.

In some implementations, the seal includes a body portion; a flange portion at a first end portion of the body portion; a sealing portion at a second end portion of the body portion; and an opening extending through the body portion, from the first end portion to the second end portion of the body portion, and the seal retention mechanism includes a recessed portion formed in an upper portion of the base plate and configured to receive a lower portion of the flange portion of the seal; a support plate coupled to the upper portion of the base plate and configured to engage an upper portion of the flange portion of the seal, such that the flange portion of the seal is retained between the support plate and the base plate, with the sealing portion of the seal extending through an opening in the support plate.

In some implementations, the seal includes a body portion; a first flange portion at a first end portion of the body portion; a second flange portion at a second end portion of the body portion, the second flange portion defining a sealing portion of the seal; and an opening extending through the first flange portion, the body portion, and the second flange portion, and the seal retention mechanism includes an undercut portion of the body portion of the base plate positioned against an outer peripheral portion of the body portion of the seal, and engaging the first flange portion and the second flange portion.

In some implementations, the seal includes a first seal portion fixed on a stepped portion of the base plate, the stepped portion extending into the at least one fluid passageway from a body portion of the base plate; and a second seal portion fixed to the diaphragm, wherein in the closed state of the diaphragm relative to the base plate, the second seal portion is positioned against the first seal portion of the seal to block an opening formed in the first seal portion and close the at least one fluid passageway; and in the open state of the diaphragm relative to the base plate, the second scal portion is spaced apart from the first seal portion of the seal, so as to open the opening formed in the first seal portion and open the at least one fluid passageway.

In some implementations, the fluid control device is one of a valve device, a pump device, or a combined pump and valve device.

In another general aspect, an implantable fluid operated inflatable device, includes a fluid control system configured to control fluid flow between a fluid reservoir and an inflatable member, the fluid control system including at least one fluid passageway within a housing; and at least one fluidic component positioned in the at least one fluid passageway and configured to control a flow of fluid through the at least one fluid passageway, the at least one fluidic component including a fluid chamber; a seal positioned between the at least one fluid passageway and the fluid chamber, and configured to seal an interface between the at least one fluid passageway and the fluid chamber in a closed position of the at least one fluidic component; and a seal retention mechanism configured to retain a position of the seal in an open position of the at least one fluidic component.

In some implementations, the seal includes an O-ring positioned in a recess surrounding an opening in a base plate of the at least one fluidic component; and the seal retention mechanism includes a texturing layer formed on a surface of the recess; and a bonding layer between the texturing layer and the O-ring, the bonding layer coupling the O-ring in the recess via the texturing layer.

In some implementations, the seal is insert-molded into a base plate of the at least one fluidic component, the seal including a body portion; a flange portion at a first end portion of the body portion; a sealing portion at a second end portion of the body portion; and an opening formed in the body portion, and the seal retention mechanism includes an undercut portion formed in a body portion of the base plate and configured to engage the flange portion of the seal; and molding material received through the opening in the body portion of the seal and integrally formed between a body portion and a central opening of the base plate.

In some implementations, the seal includes a body portion; a flange portion at a first end portion of the body portion; a sealing portion at a second end portion of the body portion; and an opening extending through the body portion, from the first end portion to the second end portion of the body portion, and the seal retention mechanism includes an undercut portion formed in a body portion of a base plate of the at least one fluidic component and configured to engage an upper portion of the flange portion of the seal; and a support plate coupled to a bottom portion of the base plate and configured to engage a bottom portion of the flange portion of the seal, such that the flange portion of the seal is retained between the base plate and the support plate.

In some implementations, the seal includes a body portion; a first flange portion at a first end portion of the body portion; a second flange portion at a second end portion of the body portion, the second flange portion defining a sealing portion of the seal; and an opening extending through the first flange portion, the body portion, and the second flange portion, and the seal retention mechanism includes an undercut portion of a base plate of the at least one fluidic component, wherein the undercut portion is positioned against an outer peripheral portion of the body portion of the seal, and engages the first flange portion and the second flange portion of the seal to retain a position of the seal relative to the base plate.

In another general aspect, an implantable fluid operated inflatable device includes a fluid control system configured to control fluid flow between a fluid reservoir and an inflatable member, the fluid control system including at least one fluid passageway within a housing; and at least one fluidic component positioned in the at least one fluid passageway and configured to control a flow of fluid through the at least one fluid passageway, the at least one fluidic component including a fluid chamber; a seal positioned between the at least one fluid passageway and the fluid chamber, and configured to seal an interface between the at least one fluid passageway and the fluid chamber in a closed position of the at least one fluidic component; and a seal retention mechanism configured to retain a position of the seal in an open position of the at least one fluidic component.

In some implementations, the at least one fluidic component includes a valve device, the valve device including a base plate; an opening formed in the base plate, connecting the fluid chamber to the at least one fluid passageway; a diaphragm coupled to the base plate, wherein the fluid chamber is defined between the base plate and the diaphragm; and a piezoelectric element coupled to the diaphragm, wherein in response to a first voltage applied to the piezoelectric element, the diaphragm is in a closed position relative to the base plate, with the seal sealing an interface between the at least one fluid passageway and the fluid chamber, and in response to a second voltage applied to the piezoelectric element, the diaphragm is deformed to an open position relative to the base plate to open the interface between the at least one fluid passageway and the fluid chamber, and the seal retention mechanism retains a position of the seal relative to the base plate.

In some implementations, the seal includes an O-ring positioned in a recess surrounding the opening formed in the base plate, and the seal retention mechanism includes a texturing layer formed on a surface of the recess; and a bonding layer between the texturing layer and the O-ring, the bonding layer coupling the O-ring in the recess via the texturing layer.

In some implementations, the seal includes an O-ring positioned in a recess surrounding the opening formed in the base plate, and the seal retention mechanism includes an undercut portion formed at an opening into the recess, wherein a dimension of the opening is less than a corresponding dimension of the O-ring such that the O-ring is retained in the recess by the undercut portion.

In some implementations, wherein the seal is an elastomer O-ring, and wherein the seal retention mechanism includes a recess surrounding the opening formed in the base plate, with the O-ring received in the recess, wherein a dimension of an inner peripheral portion of the recess is greater than a corresponding dimension of the O-ring, such that the O-ring is retained in the recess by a compression force exerted by the O-ring on the inner peripheral portion of the recess, or a dimension of an outer peripheral portion of the recess is less than a corresponding dimension of the O-ring, such that the O-ring is retained in the recess by a tensile force exerted by the O-ring on the outer peripheral portion of the recess.

In some implementations, the seal is insert-molded into the base plate, the seal including a body portion; a flange portion at a first end portion of the body portion; a scaling portion at a second end portion of the body portion; and an opening formed in the body portion, and the seal retention mechanism includes an undercut portion formed in a body portion of the base plate and configured to engage the flange portion of the seal; and molding material received through the opening in the body portion of the seal and integrally formed between a body portion and a central opening of the base plate.

In some implementations, the seal includes a body portion; a flange portion at a first end portion of the body portion; a sealing portion at a second end portion of the body portion, the sealing portion extending through a corresponding opening in the base plate; and an opening extending through the body portion, from the first end portion to the second end portion of the body portion, and the seal retention mechanism includes an undercut portion formed in a body portion of the base plate and configured to engage an upper portion of the flange portion of the seal; and a support plate coupled to a bottom portion of the base plate and configured to engage a bottom portion of the flange portion of the seal, such that the flange portion of the seal is retained between the base plate and the support plate.

In some implementations, the seal includes a body portion; a flange portion at a first end portion of the body portion; a sealing portion at a second end portion of the body portion; and an opening extending through the body portion, from the first end portion to the second end portion of the body portion, and the seal retention mechanism includes a recessed portion formed in an upper portion of the base plate and configured to receive a lower portion of the flange portion of the seal; a support plate coupled to the upper portion of the base plate and configured to engage an upper portion of the flange portion of the seal, such that the flange portion of the seal is retained between the support plate and the base plate, with the sealing portion of the seal extending through an opening in the support plate.

In some implementations, the seal includes a body portion; a first flange portion at a first end portion of the body portion; a second flange portion at a second end portion of the body portion, the second flange portion defining a sealing portion of the seal; and an opening extending through the first flange portion, the body portion, and the second flange portion, and the seal retention mechanism includes an undercut portion of the body portion of the base plate positioned against an outer peripheral portion of the body portion of the seal, and engaging the first flange portion and the second flange portion.

In some implementations, the seal includes a first seal portion fixed on a stepped portion of the base plate, the stepped portion extending into the at least one fluid passageway from a body portion of the base plate; and a second seal portion fixed to the diaphragm, wherein in the closed position of the diaphragm relative to the base plate, the second seal portion is positioned against the first seal portion of the seal to block an opening formed in the first seal portion and close the at least one fluid passageway; and in the open position of the diaphragm relative to the base plate, the second seal portion is spaced apart from the first seal portion of the seal, so as to open the opening formed in the first seal portion and open the at least one fluid passageway.

In another general aspect, a fluid control system for an implantable fluid operated inflatable device includes at least one fluid passageway defined within a housing; and a fluid control device positioned in the at least one fluid passageway, the fluid control device including a base plate; a diaphragm coupled to the base plate; a fluid chamber defined between the base plate and the diaphragm; an opening formed in the base plate, the opening connecting the fluid chamber to a fluid passageway of the fluid control device; a seal provided at the opening and configured to form a seal between the fluid passageway and the fluid chamber in a closed state of the fluid control device; and a seal retention mechanism engaged with the seal and configured to retain a position of the seal in an open state of the fluid control device.

In some implementations, the seal includes an O-ring positioned in a recess surrounding the opening formed in the base plate; and the seal retention mechanism includes a texturing layer formed on a surface of the recess; and a bonding layer between the texturing layer and the O-ring, the bonding layer coupling the O-ring in the recess via the texturing layer.

In some implementations, the seal includes an elastomer O-ring received in a recess surrounding the opening formed in the base plate, and the seal retention mechanism includes an undercut portion formed at an opening into the recess, wherein a dimension of the opening is less than a corresponding dimension of the elastomer O-ring such that the elastomer O-ring is retained in the recess by the undercut portion.

In some implementations, the seal is an elastomer O-ring, and wherein the seal retention mechanism includes a recess surrounding the opening formed in the base plate, with the elastomer O-ring received in the recess, wherein a dimension of an inner peripheral portion of the recess is greater than a corresponding dimension of the O-ring, such that the O-ring is retained in the recess by a compression force exerted by the O-ring on the inner peripheral portion of the recess, or a dimension of an outer peripheral portion of the recess is less than a corresponding dimension of the O-ring, such that the O-ring is retained in the recess by a tensile force exerted by the O-ring on the outer peripheral portion of the recess.

In some implementations, the seal is insert-molded into the base plate, the seal including a body portion; a flange portion at a first end portion of the body portion; a sealing portion at a second end portion of the body portion; and an opening formed in the body portion, and the seal retention mechanism includes an undercut portion formed in a body portion of the base plate and configured to engage the flange portion of the seal; and molding material received through the opening in the body portion of the seal and integrally formed between a body portion and a central opening of the base plate.

In some implementations, the seal includes a body portion; a flange portion at a first end portion of the body portion; a sealing portion at a second end portion of the body portion, the sealing portion extending through a corresponding opening in the base plate; and an opening extending through the body portion, from the first end portion to the second end portion of the body portion, and the seal retention mechanism includes an undercut portion formed in a body portion of the base plate and configured to engage an upper portion of the flange portion of the seal; and a support plate coupled to a bottom portion of the base plate and configured to engage a bottom portion of the flange portion of the seal, such that the flange portion of the seal is retained between the base plate and the support plate.

In some implementations, the seal includes a body portion; a flange portion at a first end portion of the body portion; a sealing portion at a second end portion of the body portion; and an opening extending through the body portion, from the first end portion to the second end portion of the body portion, and the seal retention mechanism includes a recessed portion formed in an upper portion of the base plate and configured to receive a lower portion of the flange portion of the seal; a support plate coupled to the upper portion of the base plate and configured to engage an upper portion of the flange portion of the seal, such that the flange portion of the seal is retained between the support plate and the base plate, with the sealing portion of the seal extending through an opening in the support plate.

In some implementations, the seal includes a body portion; a first flange portion at a first end portion of the body portion; a second flange portion at a second end portion of the body portion, the second flange portion defining a sealing portion of the seal; and an opening extending through the first flange portion, the body portion, and the second flange portion, and the seal retention mechanism includes an undercut portion of the body portion of the base plate positioned against an outer peripheral portion of the body portion of the seal, and engaging the first flange portion and the second flange portion.

In some implementations, the seal includes a first seal portion fixed on a stepped portion of the base plate, the stepped portion extending into the at least one fluid passageway from a body portion of the base plate; and a second seal portion fixed to the diaphragm, wherein in the closed state of the diaphragm relative to the base plate, the second seal portion is positioned against the first seal portion of the seal to block an opening formed in the first seal portion and close the at least one fluid passageway; and in the open state of the diaphragm relative to the base plate, the second seal portion is spaced apart from the first seal portion of the seal, so as to open the opening formed in the first seal portion and open the at least one fluid passageway.

In some implementations, the fluid control device is one of a valve device, a pump device, or a combined pump and valve device.

### BRIEF DESCRIPTION OF THE DRAWINGS

FIG. 1 is a block diagram of an implantable fluid operated inflatable device according to an aspect.

FIG. 2 illustrates a system including an example implantable fluid operated inflatable device according to an aspect.

FIG. 3A is a schematic diagram of a fluidic architecture of an implantable fluid operated inflatable device according to an aspect.

FIG. 3B is a schematic diagram of a fluidic architecture of an implantable fluid operated inflatable device according to an aspect.

FIG. 4A is an exploded view of an example valve device of a fluid control system of a fluid operated inflatable device according to an aspect.

FIG. 4B is a cross-sectional view of the example valve device shown in FIG. 4A, in a closed position.

FIG. 4C is a cross-sectional view of the example valve device shown in FIG. 4A, in an open position.

FIG. 5 illustrates an example seal retention device, according to an aspect.

FIG. 6 illustrates an example seal retention device, according to an aspect.

FIG. 7A illustrates an example seal retention device, according to an aspect.

FIG. 7B illustrates an example seal retention device, according to an aspect.

FIG. 8A is a perspective view of an example seal retention device, according to an aspect.

FIG. 8B is a perspective view of an example seal of the example seal retention device shown in FIG. 8A.

FIG. 8C is a cross-sectional view taken along line A-A of FIG. 8A.

FIG. 8D is a cross-sectional view taken along line B-B of FIG. 8A.

FIG. 9A is a perspective view of an example base plate and an example seal, according to an aspect.

FIG. 9B is an exploded perspective view of the example base plate and the example seal shown in FIG. 9A.

FIG. 9C is a cross-sectional view, taken along line E-E of FIG. 9A.

FIG. 10A is a perspective view of an example base plate and an example seal, according to an aspect.

FIG. 10B is an exploded perspective view of the example base plate and

the example seal shown in FIG. 11A.

FIG. 10C is a cross-sectional view, taken along line F-F of FIG. 10A.

FIG. 11A is a perspective view of an example base plate and an example seal, according to an aspect.

FIG. 11B is a perspective view of the example seal shown in FIG. 11A.

FIG. 11C is a cross-sectional view, taken along line G-G of FIG. 11A.

FIG. 12A is a cross-sectional view of an example seal incorporated into an example valve device, in a closed position, according to an aspect.

FIG. 12B is a cross-sectional view of the example seal incorporated into the example valve device, in an open position, according to an aspect.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the implementations are directed to bodily implants. The term patient or user may hereinafter be used for a person who benefits from the medical device or the methods disclosed in the present disclosure. For example, the patient can be a person whose body is implanted with the medical device or the method disclosed for operating the medical device by the present disclosure.

An implantable fluid operated inflatable device may include a fluid control system. In some examples, the fluid control system includes at least one pump and/or at least one valve and/or at least one combined pump and valve device. In some examples, the components of the fluid control system control the flow of fluid between a fluid reservoir and an inflatable member of the implantable fluid operated inflatable device, to provide for the inflation/pressurization and deflation/depressurization of the inflatable member. In some examples, the components of the fluid control system include at least one seal that selectively provides for the closing and/or opening of fluid passageways between the reservoir and the inflatable member. An implantable fluid operated inflatable device, in accordance with implementations described herein, includes at least one retention device incorporated into at least one of the components of the fluid control system, to retain the at least one seal in a seated position relative to the component in which the at least one seal is installed. In some examples, the retention mechanism includes an undercut in the recess in which the seal is installed, to retain the seal in the recess. In some examples, the retention mechanism includes a configuration of the recess that puts the seal in tension, to retain the seal in the recess. In some examples, the retention mechanism includes a configuration of the recess that puts the seal in compression, to retain the seal in the recess. In some examples, the retention mechanism includes adhesive layer that interacts with an adhesion surface in the recess and a corresponding surface of the seal, to retain the seal in the recess. In some examples, the retention mechanism includes at least one flange formed at an end portion of the seal, that interacts with a corresponding surface of a base portion of the component in which the seal is installed, to retain a position of the seal relative to the base portion of the component.

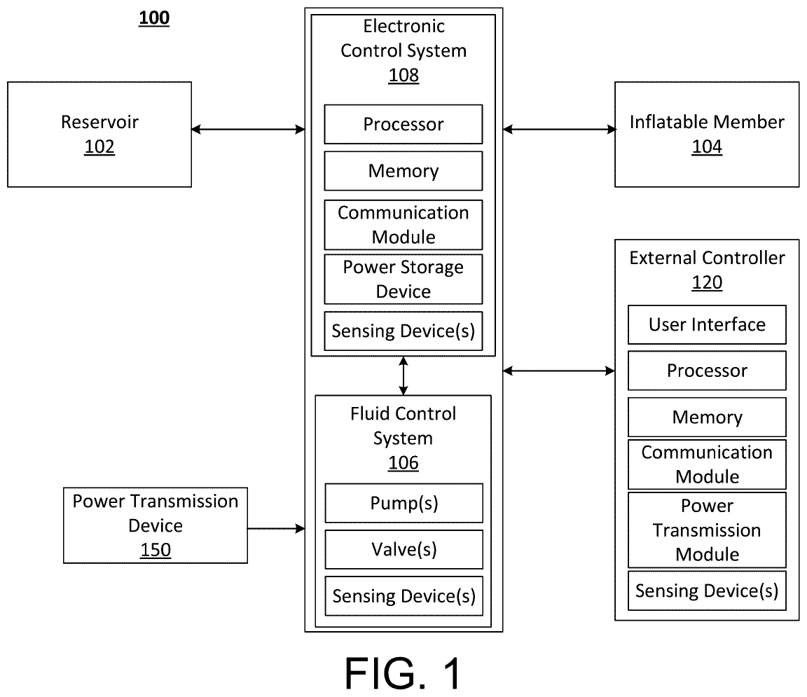

FIG. 1 is a block diagram of an example implantable fluid operated inflatable device 100. The example inflatable device 100 shown in FIG. 1 includes a fluid reservoir 102, an inflatable member 104, and an electronic control system 108. The electronic control system 108 may interface with a fluid control system 106. The fluid control system 106 can include fluidics components such as one or more pumps, one or more valves and the like configured to transfer fluid between the fluid reservoir 102 and the inflatable member 104. The fluid control system 106 can include one or more sensing devices that sense conditions such as, for example, fluid pressure, fluid flow rate and the like within the fluidics architecture of the inflatable device 100. In some implementations, the electronic control system 108 includes components that provide for the monitoring and/or control of the operation of various fluidics components of the fluid control system 106 and/or communication with one or more sensing device(s) within the implantable fluid operated inflatable device 100 and/or communication with one or more external device(s). In some examples, the electronic control system 108 includes components such as a processor, a memory, a communication module, a power storage device, or battery, sensing devices such as, for example an accelerometer, and other such components configured to provide for the operation and control of the implantable fluid operated inflatable device 100. In some examples, the communication module of the electronic control system 108 may provide for communication with one or more external devices such as, for example, an external controller 120.

In some examples, the external controller 120 includes components such as, for example, a user interface, a processor, a memory, a communication module, a power transmission module, and other such components providing for operation and control of the external controller 120 and communication with the electronic control system 108 of the inflatable device 100. For example, the memory may store instructions, applications and the like that are executable by the processor of the external controller 120. The external controller 120 may be configured to receive user inputs via, for example, the user interface, and to transmit the user inputs, for example, via the communication module, to the electronic control system 108 for processing, operation and control of the inflatable device 100. Similarly, the electronic control system 108 may, via the respective communication modules, transmit operational information to the external controller 120. This may allow operational status of the inflatable device 100 to be provided, for example, through the user interface of the external controller 120, to the user, may allow diagnostics information to be provided to a physician, and the like.

In some examples, the power transmission module of the external controller 120 provides for charging of the components of the internal electronic control system 108. In some examples, transmission of power for the charging of the internal electronic control system 108 can be, alternatively or additionally, provided by an external power transmission device 150 that is separate from the external controller 120. In some implementations the external controller 120 can include sensing devices such as one or more pressure sensors, one or more accelerometers, and other such sensing devices. In some implementations, a pressure sensor in the external controller 120 may provide, for example, a local atmospheric or working pressure to the internal electronic control system 108, to allow the inflatable device 100 to compensate for variations in pressure. In some implementations, an accelerometer in the external controller 120 may provide detected patient movement to the internal electronic control system 108 for control of the inflatable device 100.

The fluid reservoir 102, the inflatable member 104, the electronic control system 108 and the fluid control system 106 may be internally implanted into the body of the patient. In some implementations, the electronic control system 108 and the fluid control system 106 are coupled in or incorporated into a housing. In some implementations, at least a portion of the electronic control system 108 is physically separate from the fluid control system 106. In some implementations, some modules of the electronic control system 108 are coupled to or incorporated into the fluid control system 106, and some modules of the electronic control system 108 are separate from the fluid control system 106. For example, in some implementations, some modules of the electronic control system 108 are included in an external device (such as the external controller 120) that is in communication other modules of the electronic control system 108 included within the implantable fluid operated inflatable device 100. In some implementations, at least some aspects of the operation of the implantable fluid operated inflatable device 100 may be manually controlled.

In some examples, electronic monitoring and control of the implantable fluid operated inflatable device 100 may provide for improved patient control of the device, improved patient comfort, improved patient safety, and the like. In some examples, electronic monitoring and control of the implantable fluid operated inflatable device 100 may afford the opportunity for tailoring of the operation of the inflatable device 100 by a physician without further surgical intervention. Fluidic architecture defining the flow and control of fluid through the implantable fluid operated inflatable device 100, including the configuration and placement of fluidics components such as pumps, valves, sensing devices and the like, may allow the inflatable device 100 to precisely monitor and control operation of the inflatable device, effectively respond to user inputs, and quickly and effectively adapt to changing conditions both within the inflatable device 100 (changes in pressure, flow rate and the like) and external to the inflatable device 100 (pressure surges due to physical activity, impacts and the like, sustained pressure changes due to changes in atmospheric conditions, and other such changes in external conditions).

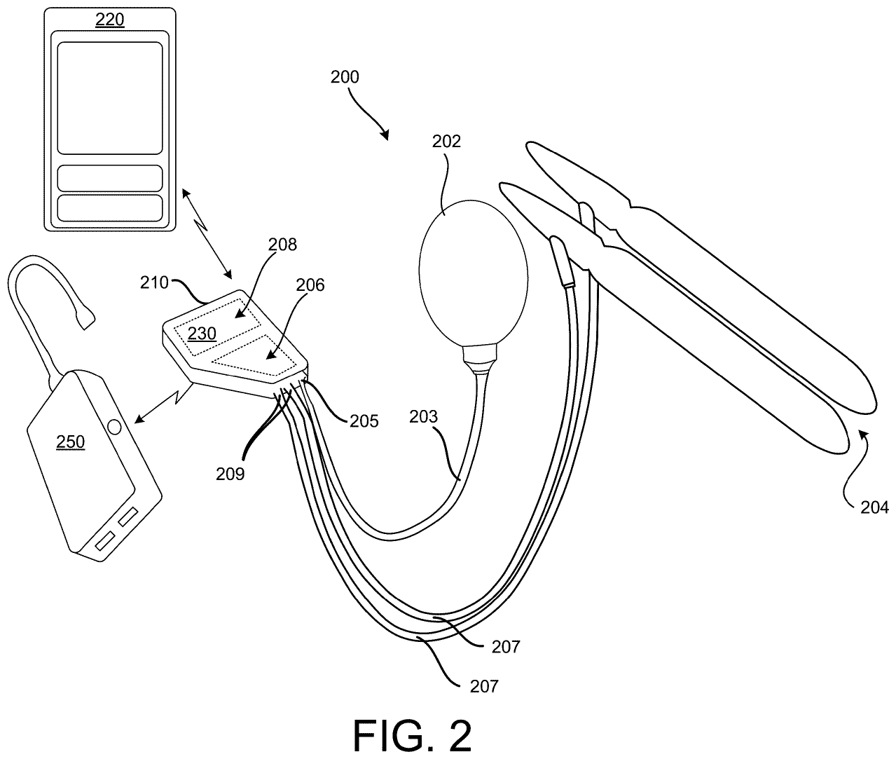

The example implantable fluid operated inflatable device 100 may be representative of a number of different types of implantable fluid operated devices. For example, the device 100 shown in FIG. 1 may be representative of an inflatable penile prosthesis as shown in FIG. 2. In some implementations, the example implantable fluid operated inflatable device 100 shown in FIG. 1 may be representative of other types of implantable inflatable devices that rely on the control of fluid flow to components of the device to achieve inflation, pressurization, deflation, depressurization, deactivation, and the like, such as, for example, an artificial urinary sphincter, and other such devices.

An example system including an example implantable fluid operated inflatable device 200 in the form of an example inflatable penile prosthesis is shown in FIG. 2. The example inflatable device 200 includes a fluid control system 206 (similar to the example fluid control system 106 described above with respect to FIG. 1) including fluidics components such as pumps, valves, sensing devices and the like positioned in fluid passageways. In some implementations, the fluid control system includes components such as, for example, one or more fluid control devices, one or more pressure sensors, and other such components. In some implementations, the example inflatable device 200 includes an electronic control system 208 (similar to the example electronic control system 108 described above with respect to FIG. 1) configured to provide for the transfer of fluid between a reservoir 202 (such as the example reservoir 102 described above with respect to FIG. 1) and an inflatable member 204 (similar to the example inflatable member 104 described above with respect to FIG. 1) via the fluidics components. In the example shown in FIG. 2, the inflatable member 204 is in the form of a pair of inflatable cylinders. In the example shown in FIG. 2, fluidics components of the fluid control system 206, and electronic components of the electronic control system 208 are received in a housing 210. In some implementations, fluidics components of the fluid control system 206, and electronic components of the electronic control system 208 received in the housing 210 together define an electronically controlled fluid manifold 230 that provides for the electronic control of the flow of fluid between the reservoir 202 and the inflatable member 204.

In the example shown in FIG. 2, a first conduit 203 connects a first fluid port 205 of the electronically controlled fluid manifold 230 (the fluid control system 206/electronic control system 208 received in the housing 210) with the reservoir 202. One or more second conduits 207 connect one or more second fluid ports 209 of the electronically controlled fluid manifold 230 (the fluid control system 206/electronic control system 208 received in the housing 210) with the inflatable member 204 in the form of the inflatable cylinders. In some examples, the electronic control system 208 can communicate with an external controller 220 (similar to the external controller 120 described above with respect to FIG. 1), via respective communication modules. For example, an application stored in a memory and executed by a processor of the external controller 220 may allow the user and/or a physician to operate, view, monitor and alter operation of the inflatable device 200. In some examples, components of the electronic control system 208 and/or the fluid control system 206 can be charged and/or recharged by a power transmission module of the external controller 220, and/or by a power transmission device 250, that is separate from the external controller 220.

The principles to be described herein are applicable to the example implantable fluid operated inflatable device, in the form of the example inflatable penile prostheses shown in FIG. 2, and other types of implantable fluid operated inflatable devices that rely on a pump and valve assembly including various fluidics components to provide for the transfer of fluid between the different fluid filled implantable components to achieve inflation, deflation, pressurization, depressurization, deactivation, occlusion, and the like for effective operation. The example implantable fluid operated inflatable device 200 shown in FIG. 2 includes an electronic control system 208 to provide for control of the operation of the respective inflatable members 204 in the form of cylinders, and the monitoring and control of pressure and/or fluid flow through inflatable members 204. Some of the principles to be described herein may also be applied to implantable fluid operated inflatable devices that are manually controlled.

As noted above, the electronic control system 208 controlling the flow of fluid between the reservoir 202 and the inflatable member 204 for inflation, pressurization, deflation, depressurization and the like of the inflatable member 204 may provide for improved patient control of the inflatable device 200, improved accuracy in operation of the inflatable device 200, improved patient comfort, improved patient safety, and the like. In some situations, this improved control and improved accuracy in the operation of the inflatable device 200 may rely on precise operation and control of the components within the fluid control system 206 and/or the electronically controlled fluid manifold 230. Accordingly, in some implementations, the electronically controlled fluid manifold 230 includes a fluid control system 206 having one or more pump and/or valve devices. Accurate and consistent operation of the components of the pump and/or valve devices may produce the desired accurate flow control, and consistent inflation, deflation, pressurization, depressurization, deactivation, occlusion, and the like for effective operation.

A fluid control system, in accordance with implementations described herein, can include a pump assembly including, for example, one or more pump devices and valve devices and/or combined pump and valve devices within a fluid circuit of the pump assembly to control the transfer fluid between the fluid reservoir and the inflatable member. In some examples, the pump assembly including the one or more pump devices and valve device(s) and/or combined pump and valve devices is electronically controlled. In an example in which the pump assembly is electronically powered and/or controlled, the pump assembly may include a hermetic manifold that can contain and segment the flow of fluid from electronic components of the pump assembly, to prevent leakage and/or gas exchange. In some examples, the one or more pump devices and valve devices and/or combined pump and valve devices include piezoelectric elements. In some examples, the pump assembly includes one or more pressure sensing devices in the fluid circuit to provide for relatively precise monitoring and control of fluid flow and/or fluid pressure within the fluid circuit and/or the inflatable member. A fluid circuit configured in this manner may facilitate the proper inflation, deflation, pressurization, depressurization, and deactivation of the components of the implantable fluid operated device to provide for patient safety and device efficacy.

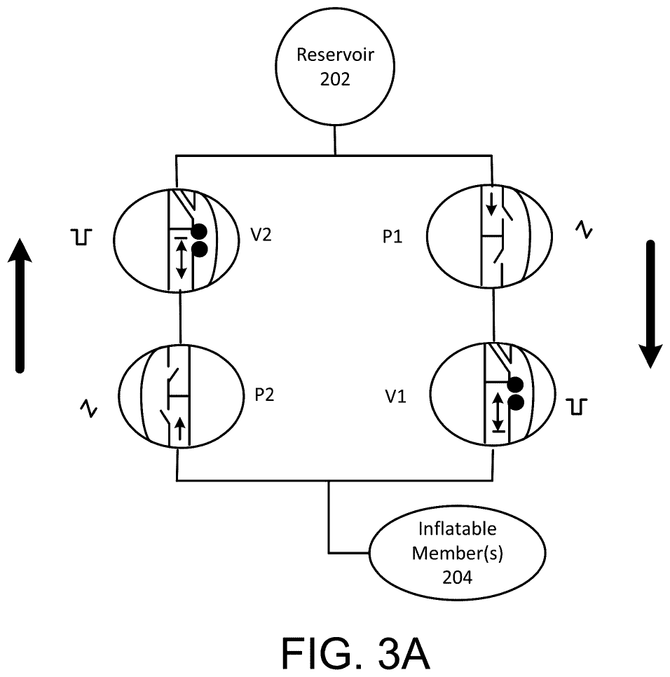

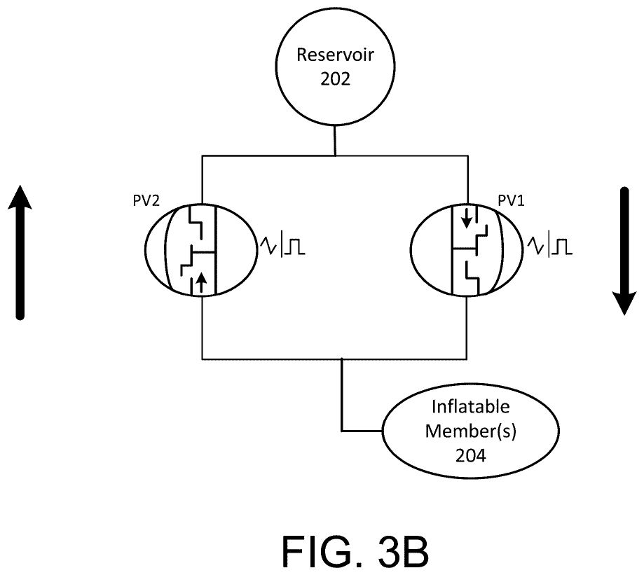

FIG. 3A is a schematic diagram of an example fluidic architecture for an implantable fluid operated inflatable device, according to an aspect. FIG. 3B is a schematic diagram of an example fluidic architecture for an implantable fluid operated inflatable device, according to an aspect. The fluidic architecture of an implantable fluid operated inflatable device can include other arrangements of fluidic channels, pump(s)/valve(s), pressure sensor(s) and other components than the examples shown in FIGS. 3A and 3B.

The example fluidic architecture shown in FIG. 3A includes a first pump P1 and a first valve V1 positioned in a first fluid passageway, between the reservoir 202 and the inflatable member 204, to control the flow of fluid from the reservoir 202 to the inflatable member 204. The example fluidic architecture shown in FIG. 3A includes a second pump P2 and a second valve V2 positioned in a second fluid passageway, between the inflatable member 204 and the reservoir 202, to control the flow of fluid from the inflatable member 204 to the reservoir 202. As shown in FIG. 3B, in some examples, the first pump and the first valve are included in a combination pump and valve device PV1 provided in the first fluid passageway, and the second pump and the second valve are included in a second combination pump and valve device PV2 provided in the second fluid passageway. The fluidic architecture of an implantable fluid operated inflatable device can include other arrangements of fluidic channels, pump(s)/valve(s), pressure sensor(s) and other components than shown in FIGS. 3A and 3B.

In example fluidic architecture shown in FIG. 3A, the first pump P1 and the first valve V1 operate to pump fluid from the reservoir 202 to the inflatable member 204 through the first fluid passageway to provide for inflation of the inflatable member 204, while the second valve V2 closes the second fluid passageway to provide to prevent backflow of fluid, back to the reservoir 202. The second pump P2 and the second valve V2 operate to pump fluid from the inflatable member 204 to the reservoir 202 through the second fluid passageway to provide for deflation of the inflatable member 204, while the first valve V1 closes the first fluid passageway to prevent backflow of fluid to the inflatable member 204.

In the example arrangement shown in FIG. 3B, the first combined pump and valve device PV1 and the second combined pump and valve device PV2 may be operated in a first mode to inflate or pressurize the inflatable member 204, and in a second mode to deflate or repressurize the inflatable member 204. In the first mode of operation, the first combined pump and valve device PV1 convey fluid from the reservoir 202 to the inflatable member 204, while the second combined pump and valve device PV2 remains closed/inoperable to prevent flow of fluid from the inflatable member 204 towards the reservoir 202 to prevent deflation/depressurization. The first combined pump and valve device PV1 may remain operable to pump fluid to the inflatable member 204 until a desired pressure is achieved. The first combined pump and valve device PV1 may be closed once the desired pressure is achieved, to maintain the inflatable member 204 at the desired pressure/inflated state. In the second mode of operation, the second combined pump and valve device PV2 convey fluid from the inflatable member 204 to the reservoir 202, while the first combined pump and valve device PV1 remains closed/inoperable to prevent flow of fluid from the reservoir 202 towards the inflatable member 204 to prevent inflation/pressurization. The second combined pump and valve device PV2 may remain operable to pump fluid to the reservoir 202 until a desired pressure is achieved at the inflatable member 204. The second combined pump and valve device PV2 may be closed once the desired pressure is achieved, to maintain the inflatable member 204 at the desired pressure/in the deflated state.

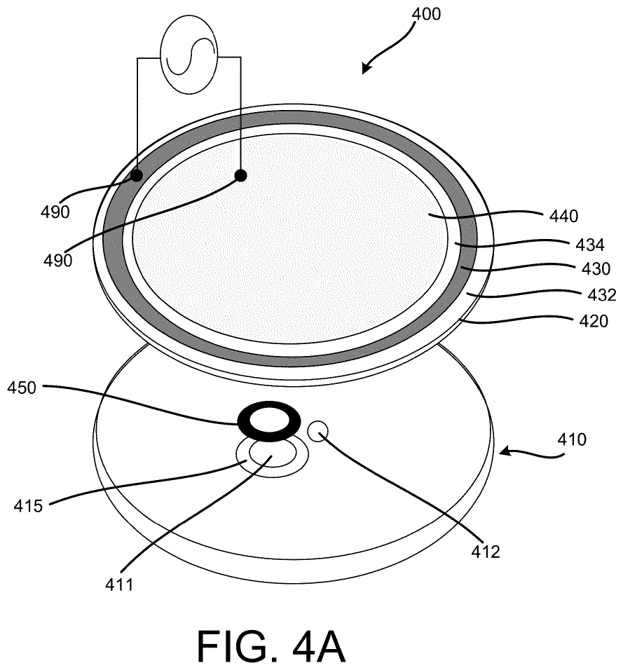

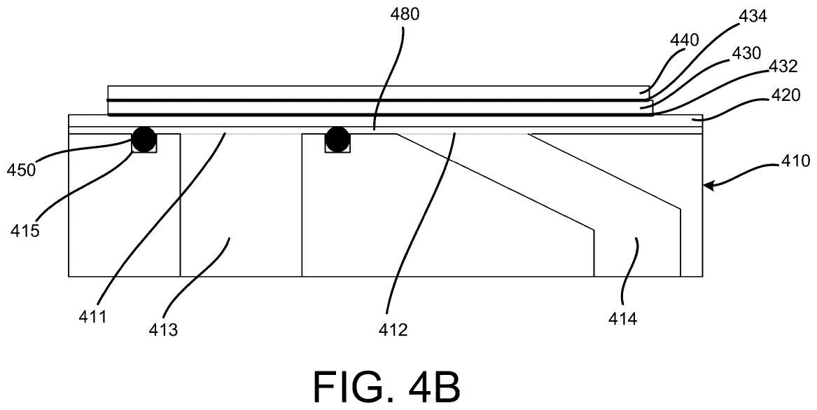

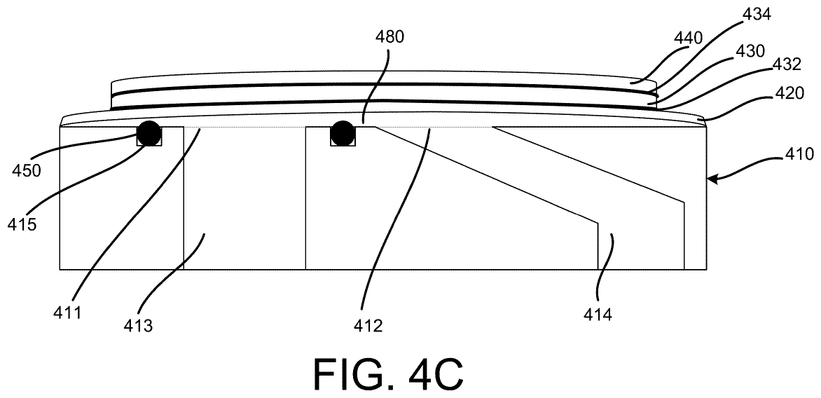

FIG. 4A is a partially exploded perspective view of an example valve device 400, in accordance with implementations described herein. FIGS. 4B and 4C are cross-sectional views of the example valve device 400 shown in FIG. 4A, in an assembled state, illustrating operation of the example valve device 400. The example valve device 400 shown in FIGS. 4A-4C is an example of a fluid control device, or a fluidic component, that may be included in the fluid control system 206 of the example electronically controlled fluid manifold 230 described above.

In the example arrangement shown in FIGS. 4A-4C, the example valve device 400 includes a base plate 410 defining a base portion of the valve device 400. A diaphragm 420 is positioned on the base plate 410. A piezoelectric element 440 is positioned on the diaphragm 420, with an isolation layer 430 positioned between the diaphragm 420 and the piezoelectric element 440. In some examples, an epoxy layer 432 provides for the coupling of the isolation layer 430 on the diaphragm 420. In some examples, an epoxy layer 434 provides for the coupling of the piezoelectric element 440 on the isolation layer 430. In some examples, one or more electrodes 490 are arranged on the example valve device 400. In the example shown in FIG. 4A, the example valve device 400 includes a pair of electrodes 490 coupled between the isolation layer 430 and the piezoelectric element 440. Application of voltage to the piezoelectric element 440 causes a deflection or deformation of the piezoelectric element 440, and a corresponding deflection or deformation of the diaphragm 420 and the isolation layer 430 coupled thereto. Application of voltage in this manner causes the valve device 400 to move between the closed position shown in FIG. 4B and the open position shown in FIG. 4C. In some examples, an alternating application of voltage to the piezoelectric element 440 causes the valve device 400 to alternate between the closed position and the open position, such that the valve device 400 can also perform a pumping function.

In the example arrangement shown in FIGS. 4A-4C, a fluid chamber 480 is defined between the base plate 410 and the diaphragm 420. The base plate 410 includes a first opening 411 that provides for communication between a first fluid passageway 413 and the fluid chamber 480. The base plate 410 includes a second opening 412 that provides for communication between a second fluid passageway 414 and the fluid chamber 480. In the example arrangement shown in FIGS. 4A-4C, the base plate 410 includes a recess 415 surrounding the first opening 411, with a seal 450, in the form of an O-ring in the example shown in FIGS. 4A-4C, fitted in the recess 415.

In the closed position of the example valve device 400, a top portion of the seal 450 is pressed against the diaphragm 420, as shown in FIG. 4B. This positioning of the seal 450 against the diaphragm 420 closes off the chamber 480, and inhibits the flow of fluid through the example valve device 400, between the first fluid passageway 413 and the second fluid passageway 414 via the chamber 480. In the open position of the example valve device 400, the top portion of the seal 450 is separated from, or spaced apart from, the diaphragm 420 due to the deflection of the diaphragm 420. This positioning of the seal 450 relative to the diaphragm 420 opens the chamber 480, and allows fluid to flow through the example valve device 400, between the first fluid passageway 413 and the second fluid passageway 414 via the fluid chamber 480.

In the open position of the example valve device 400, the flow of fluid in the chamber 480, in a variety of directions, may generate vortices that impart forces on the seal 450. In some examples, forces exerted on the seal 450 in this manner may cause movement of the seal 450. In some examples, forces exerted on the seal 450 in this manner may alter a position of the seal 450 in the recess. For example, forces exerted on the seal 450 in this manner may lift the seal 450 from its seated position within the recess 415. In some situations, lifting of the seal 450 from the seated position within the recess 415 with the example valve device 400 in the open position may inhibit or obstruct the flow of fluid through chamber 480. Inhibiting or obstructing the flow of fluid through the example valve device 400 may, in turn, adversely impact the inflation/pressurization and/or deflation/depressurization of the inflatable member 204 as intended. Thus, the impeded flow of fluid through the example valve device 400 may adversely impact overall effective operation of the implantable fluid operated inflatable device in which it is installed, and may adversely impact patient comfort and safety.

A valve device, in accordance with implementations described herein, includes a seal retention mechanism. The seal retention mechanism retains a position of the seal relative to the component in which it is installed, to preserve the functionality of the component as intended. Hereinafter, example retention mechanisms will be described with respect to the example valve device 400 described above, simply for purposes of discussion and illustration. The principles to be described herein are applicable to other components, devices and the like which would benefit from positive retention of a seal.

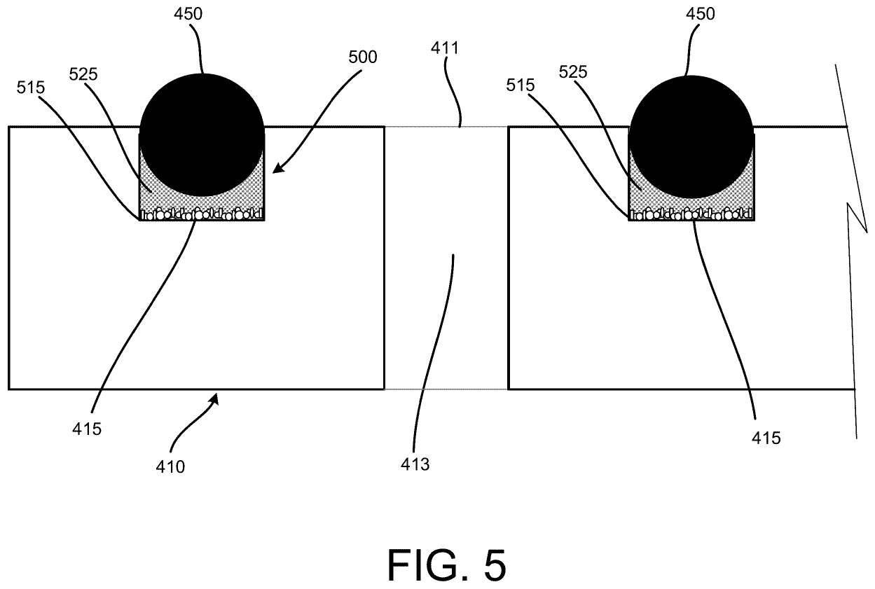

FIG. 5 illustrates an example seal and seal retention mechanism 500, in accordance with implementations described herein. Hereinafter, the example seal retention mechanism 500 will be described with respect to incorporation into the example valve device 400 described above, simply for purposes of discussion and illustration. The principles to be described with respect to the example seal retention mechanism 500 are applicable to other fluid control component which would benefit from the positive retention of a seal such as, for example, other valve devices, pump devices, combined pump and valve devices, and the like.

FIG. 5 is a close-in view of the seal 450, in the form of an O-ring, received in the recess 415 surrounding the first opening 411/first fluid passageway 413 formed in the base plate 410. In the example shown in FIG. 5, the seal retention mechanism 500 includes a texture layer 515 formed along a surface portion of the recess 415, and a bonding layer 525 provided between the texture layer 515 and a corresponding seating surface of the seal 450. In some examples, the texture layer 515 provides an erratic, or non-uniform surface to facilitate bonding and adhesion with the bonding layer 525. In some examples, the bonding layer 525 includes an adhesive material such as, for example, an epoxy material, a resin material, and the like that will form a bond between the seal 450 and the texture layer 515 in the recess 415 and retain the seal 450 in the seated position within the recess 415. In some examples, the texture layer 515 is formed by, for example, a laser ablation process applied to a surface portion of the recess 415, a machining process applied to a surface portion of the recess 415, or other such process. In some examples, the erratic or non-uniform texture of the texture layer 515 includes nodules and/or fissures and/or undercuts and/or pores and the like. In some examples, the texturing provided by these features of the texture layer 515 facilitates mechanical adhesion with a material of the bonding layer 525. In the example shown in FIG. 5, the texture layer 515 is formed on a lower surface of the recess 415 (in the example orientation shown in FIG. 5), simply for purposes of discussion and illustration. In some examples, the texture layer 515 is formed on other surface portions of the recess 415, instead of, or in addition to, the bottom surface of the recess 415. In some examples, a corresponding coupling surface, or bonding surface of the seal 450 may be modified (now shown in FIG. 5), to enhance bonding of the seal 450 with the bonding layer 525.

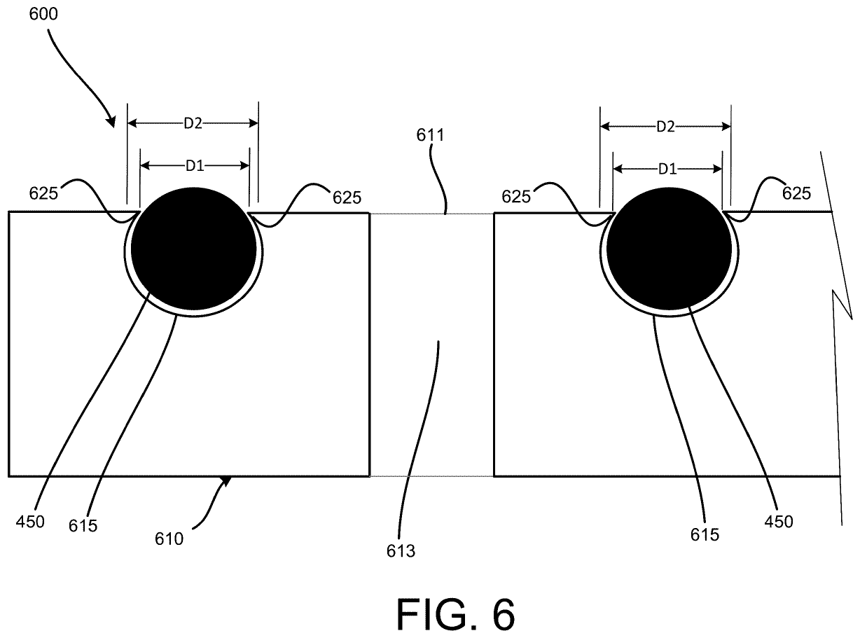

FIG. 6 illustrates an example seal and seal retention mechanism 600, in accordance with implementations described herein. Hereinafter, the example seal retention mechanism 600 will be described with respect to incorporation into a base plate of an example valve device, such as the example valve device 400 described above, simply for purposes of discussion and illustration. The principles to be described with respect to the example seal retention mechanism 600 are applicable to other fluid control components which would benefit from the positive retention of a seal such as, for example, other valve devices, pump devices, combined pump and valve devices, and the like.

FIG. 6 is a close-in view of the seal 450, in the form of an O-ring, received in a recess 615 formed in a base plate 610 of a fluidic component such as, for example, the example valve device 400 described above, or other such fluidic component. The recess 615 surrounds an opening 611 formed in the base plate 610, connecting a fluid chamber (not shown in FIG. 6) to a fluid passageway 613 (similar to the first opening 411 in the base plate 410, connecting the chamber 480 to the fluid passageway 413 of the example valve device 400). In the example shown in FIG. 6, the seal retention mechanism 600 is provided through the engagement, or interaction of an undercut portion 625 of the recess 615 with the seal 450, in the form of an O-ring. That is, a dimension D1 of the opening into the recess 615, defined at the undercut portion 625 of the recess 615 is less than a corresponding dimension D2 of the seal 450. In this example, the seal 450 is made of a compliant material, for example, an elastomer material, that deforms to fit through the opening defined by the undercut portion 625 of the recess 615. After insertion of the seal 450 into the recess 615 through the opening defined by the undercut portion 625, the seal 450 returns to its original shape/configuration. In this state, the seal 450 is retained in the recess 615 by the undercut portion 625 of the recess 615, and in particular, by the smaller dimension D1 of the undercut portion 625 of the recess 615 compared to the corresponding dimension D2 of the seal 450.

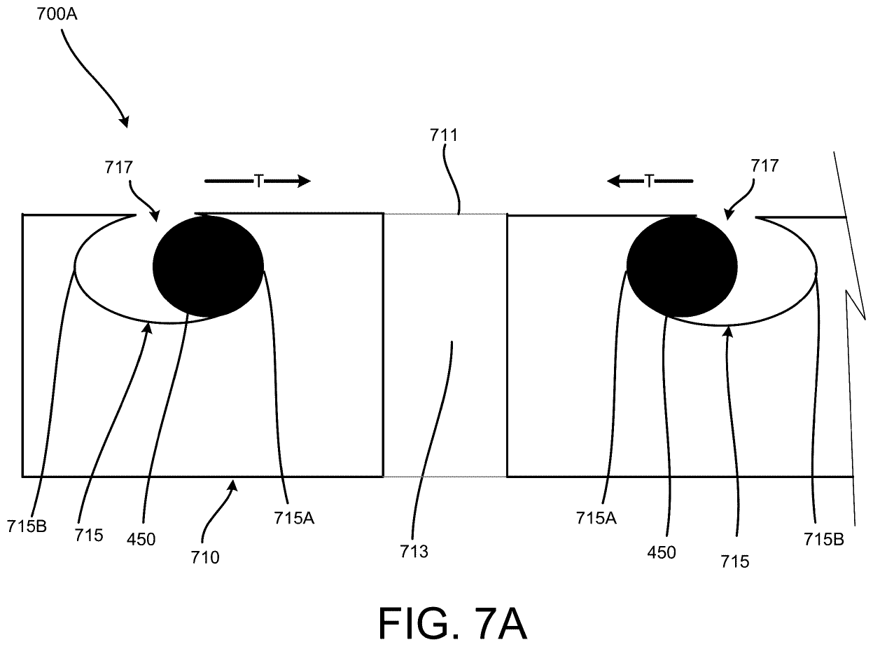

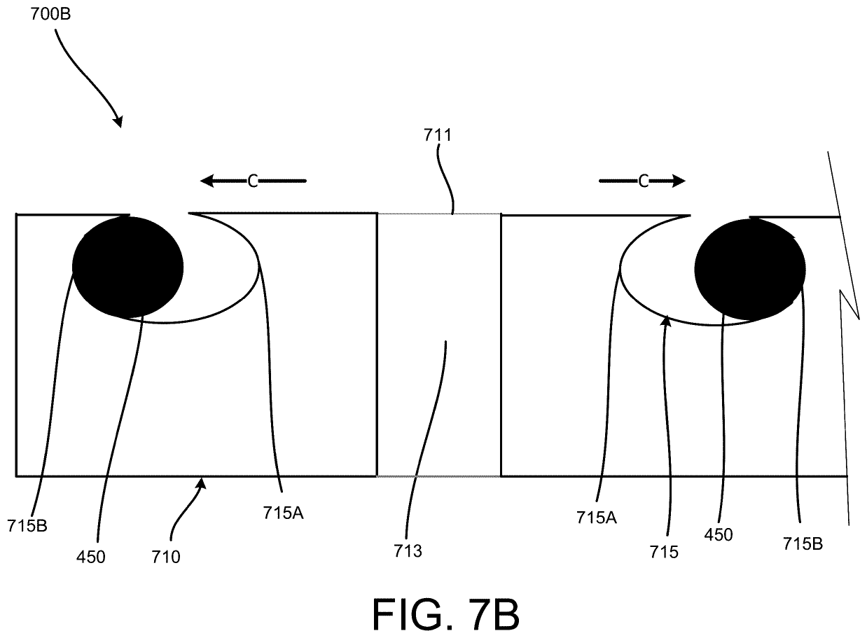

FIG. 7A illustrates an example seal and seal retention mechanism 700A, in accordance with implementations described herein. Hereinafter, the example seal retention mechanism 700A will be described with respect to incorporation into a base plate of an example valve device, such as the example valve device 400 described above, simply for purposes of discussion and illustration. The principles to be described with respect to the example seal retention mechanism 700A are applicable to other fluid control components which would benefit from the positive retention of a seal such as, for example, other valve devices, pump devices, combined pump and valve devices, and the like.

FIG. 7A is a close-in view of the seal 450, in the form of an O-ring, received in a recess 715 formed in a base plate 710 of a fluidic component such as, for example, the example valve device 400 described above, or other such fluidic component. The recess 715 surrounds an opening 711 formed in the base plate 710, connecting a fluid chamber (not shown in FIG. 7A) to a fluid passageway 713 (similar to the first opening 411 in the base plate 410, connecting the chamber 480 to the fluid passageway 413 of the example valve device 400). In the example shown in FIG. 7A, the seal retention mechanism 700A is provided by the interaction of the seal 450 with an inner peripheral portion 715A of the recess 715. For example, in an example arrangement in which the recess 715 is a substantially circular recess formed in the base plate 710 surrounding the opening 711, the inner peripheral portion 715A of the recess 715 may correspond to an inner diameter portion of the recess 715, or an inner circumferential portion of the recess 715. In the arrangement shown in FIG. 7A, the elasticity of the seal 450, for example, the elastomer material of the seal 450, allows the seal 450 to be pulled or stretched for insertion into the recess 715 through an open upper portion 717 of the recess 715. Once inserted into the recess 715, the elasticity of the seal 450 causes the seal 450 to retract, or pull inwards, in the direction of the arrows T shown in FIG. 7A. For example, a dimension, for example, a diameter, of the seal 450 in an at rest state may be less than a corresponding dimension of the inner peripheral portion 715A of the recess 715, such that once in the recess 715, the seal 450 contracts and returns to the at rest state due to the elastic properties of the seal 450. Thus, in the arrangement shown in FIG. 7A, the seal 450 is in tension, exerting a tensile force in the direction of the arrows T on the inner peripheral portion 715A of the recess 715, and retaining the seal 450 in the recess 715. In some examples, the open upper portion 717 of the recess 715 may include undercut portions as described above with FIG. 6, to provide for additional retention of the seal 450 in the recess 715.

FIG. 7B is a close-in view of the seal 450, in the form of an O-ring, received in a recess, such as the recess 715 formed in the base plate 710 shown in FIG. 7A. The base plate 710 may be incorporated into a fluidic component such as, for example, the example valve device 400 described above, or other such fluidic component. In the example shown in FIG. 7B, the seal retention mechanism 700B is provided by the interaction of the seal 450 with an outer peripheral portion 715B of the recess 715. For example, in an example arrangement in which the recess 715 is a substantially circular recess formed in the base plate 710 surrounding the opening 711, the outer peripheral portion 715B of the recess 715 may correspond to an outer diameter portion of the recess 715, or an outer circumferential portion of the recess 715. In the arrangement shown in FIG. 7B, the elasticity of the seal 450 allows the seal 450 made of an elastomer material to be compressed or squeezed or deformed for insertion into the recess 715 through the open upper portion 717 of the recess 715. Once inserted into the recess 715, the elasticity of the seal 450 causes the seal 450 to expand, or return to its original form, exerting a compressive force on the outer peripheral portion 715B of the recess in the direction of the arrows C shown in FIG. 7B. For example, a dimension, for example, a diameter, of the seal 450 in an at rest state may be greater than a corresponding dimension of the outer peripheral portion 715B of the recess 715, such that once in the recess 715, the seal 450 expands and returns to the at rest state due to the elastic properties of the seal 450. Thus, in the arrangement shown in FIG. 7B, the seal 450 is in compression within the recess 715, exerting a force in the direction of the arrows C on the outer peripheral portion 715B of the recess 715, and retaining the seal 450 in the recess 715. In some examples, the open upper portion 717 of the recess 715 may include undercut portions as described above with FIG. 6, to provide for additional retention of the seal 450 in the recess 715.

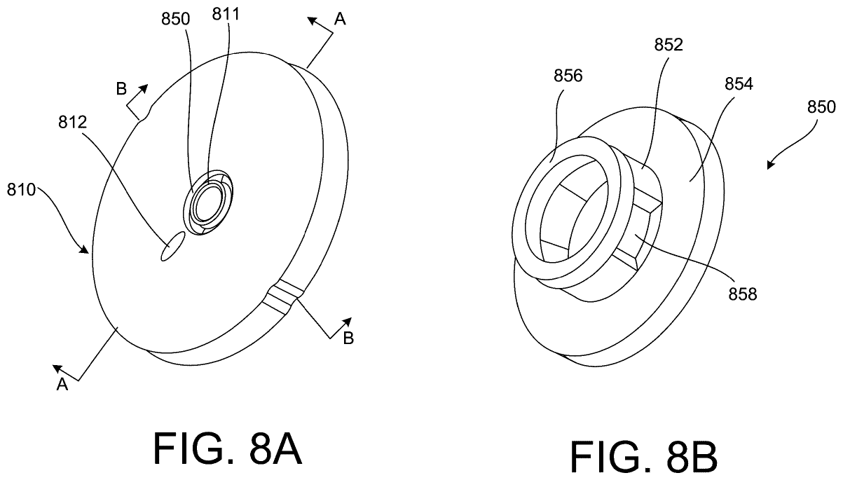

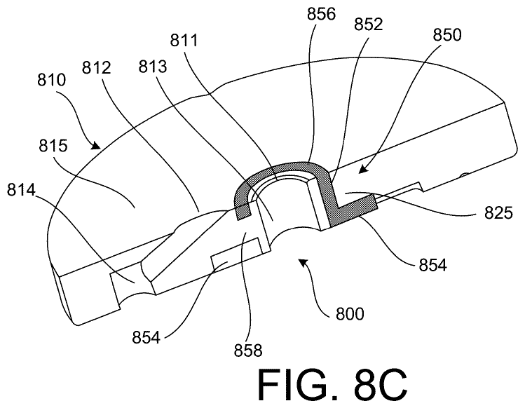

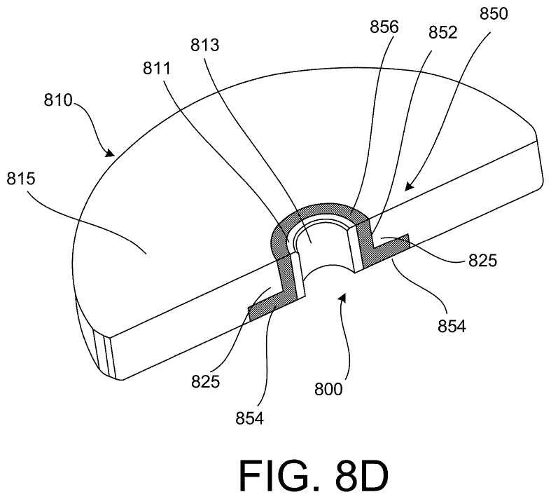

In some examples, a seal may be fabricated together with a base plate of a fluidic component. The example arrangement shown in FIGS. 8A-8D illustrates a seal that is insert molded into a base plate, with the insert molding providing positive retention of the seal relative to the base plate. Thus, FIGS. 8A-8D illustrate a retention mechanism 800 provided through insert-molding of a seal into a base plate. In particular, FIG. 8A is a perspective view of an insert molded base plate 810 and scal 850. FIG. 8B is a perspective view of the example seal 850. FIG. 8C is a cross-sectional view taken along line A-A of FIG. 8A, and FIG. 8D is a cross-sectional view taken along line B-B of FIG. 8A, illustrating the retention mechanism 800 provided by the insert molding of the seal 850 into the base plate 810.

The base plate 810 includes a first opening 811 connecting a chamber (not shown in FIGS. 8A-8D) to a first fluid passageway 813 (similar to the first opening 411 in the base plate 410, connecting the chamber 480 to the first fluid passageway 413 of the example valve device 400), a second opening 812 connecting a chamber (not shown in FIGS. 8A-8D) to a second fluid passageway 814 (similar to the second opening 412 in the base plate 410, connecting the chamber 480 to the second fluid passageway 414 of the example valve device 400). In the example shown in FIGS. 8A-8D, the seal retention mechanism 800 is provided by the positive retention of the seal 850 in the base plate 810 due to the insert molding of the seal 850 in the base plate 810.

In the example arrangement shown in FIGS. 8A-8D, the seal 850 includes a body portion 852, with a flange portion 854 extending outward, for example radially outward, from a first end portion of the body portion 852, and a scaling portion 856 provided at a second end portion of the body portion 852. In the example shown in FIGS. 8A-8D, the body portion 852 of the seal 850 surrounds the first fluid passageway 813 defined by the base plate 810. In some examples, the seal 850 includes an opening 858. In the example shown in FIGS. 8A-8D, the opening 858 is formed in the body portion 852 of the seal 850, to provide for the flow of molding material from a body portion 815 of the base plate 810 to the portion of the base plate 810 defining the first fluid passageway 813, such that the base plate 810 and the seal 850 can be integrally formed. In the example arrangement shown in FIGS. 8A-8D, the flange portion 854 of the seal 850 provides a seating surface of the seal 850 that mates with an undercut portion 825 formed in the base plate 810. The undercut portion 825 of the base plate 810 (formed by molding material received in the area of the undercut portion 825 in the molding of the base plate 810) engages the flange portion 854 of the seal 850, to form the retention mechanism 800 that positively retains the position of the seal 850 relative to the base plate 810. The seal 850 is further retained by the material received in the opening 858 in the body portion 852 of the seal 850, that integrally forms the body portion 815 of the base plate 810 and the first fluid passageway 813.

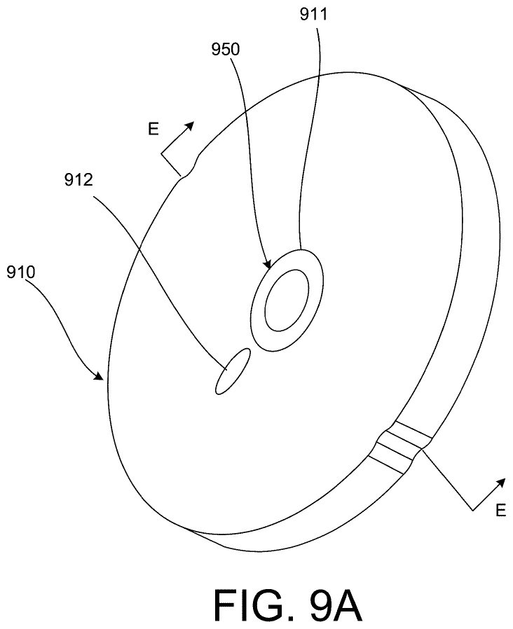

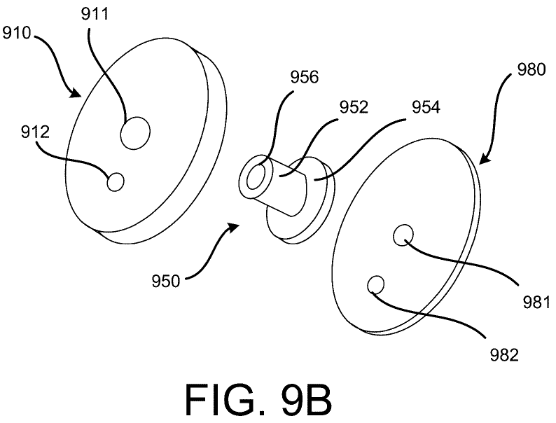

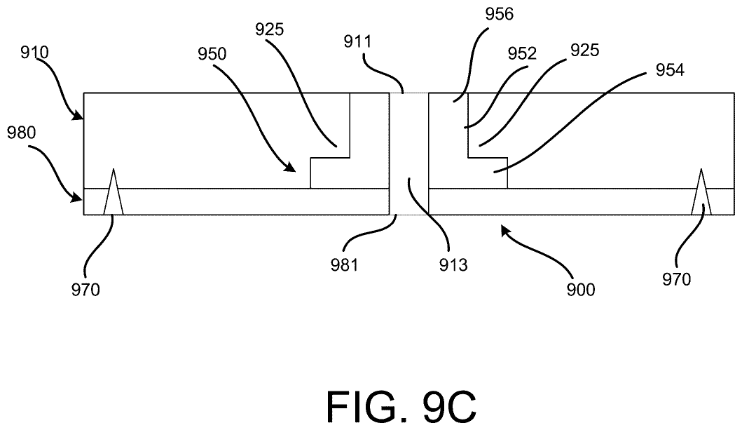

In some examples, a seal may be mechanically retained in a base plate of a fluidic component. The example arrangement shown in FIGS. 9A-9C illustrates a scal that is mechanically retained in a base plate to provide for positive retention of the seal relative to the base plate. Thus, FIGS. 9A-9C illustrate a retention mechanism 900 provided through the mechanical coupling of a seal to a base plate. In particular, FIG. 8A is a perspective view of a base plate 910 and seal 950. FIG. 9B is an exploded perspective view of the base plate 910 and seal 950 shown in FIG. 9A. FIG. 9C is a cross-sectional view, taken along line E-E of FIG. 9A, illustrating the retention mechanism 900 provided by the mechanical coupling of the seal 950 and the base plate 910.

The base plate 910 includes a first opening 911 connecting a chamber (not shown in FIGS. 9A-9C) to a first fluid passageway 913 (similar to the first opening 411 in the base plate 410, connecting the chamber 480 to the first fluid passageway 413 of the example valve device 400), and a second opening 912 connecting a chamber to a second fluid passageway (not shown in FIGS. 9A-9C). In the example shown in FIGS. 9A-9C, the seal retention mechanism 900 is provided by the positive retention of the seal 950 relative to the base plate 910 due to the attachment of a support plate 980 coupled to the base plate 910, with the seal 950 positioned therebetween.

In the example arrangement shown in FIGS. 9A-9C, the seal 950 includes a body portion 952, with a flange portion 954 extending outward, for example radially outward, from a first end portion of the body portion 952, and a sealing portion 956 provided at a second end portion of the body portion 952. In the example shown in FIGS. 9A-9C, the body portion 952 of the seal 950 is positioned in the first opening 911, and extends along an inner portion of the first fluid passageway 913. A support plate 980 is coupled to the base plate 910, with the seal 950 positioned therebetween, to provide for positive retention of the seal 950 relative to the base plate 910. In some examples, the support plate 980 includes a first opening 981 corresponding to the first opening 911 in the base plate 910, and to the first fluid passageway 913 through the body portion 952 of the seal 950, and a second opening 982 corresponding to the second opening 912 in the base plate 910. In the example shown in FIGS. 9A-9C, the support plate 980 is coupled to the base plate 910 by at least one weld 970, simply for purposes of discussion and illustration. Other coupling devices may be used to couple the support plate 980 to the base plate 910.

In the example arrangement shown in FIGS. 9A-9C, the flange portion 954 of the seal 850 provides a seating surface of the seal 950 that engages an undercut portion 925 formed in the base plate 910. This interface, between the flange portion 954 of the seal 950 and the undercut portion 925 of the base plate 910, together with the coupling of the support plate 980 to a bottom portion of the base plate 910 and a bottom portion of the flange portion 954 of the seal 950, forms the retention mechanism 900 that positively retains the position of the seal 950 relative to the base plate 910.

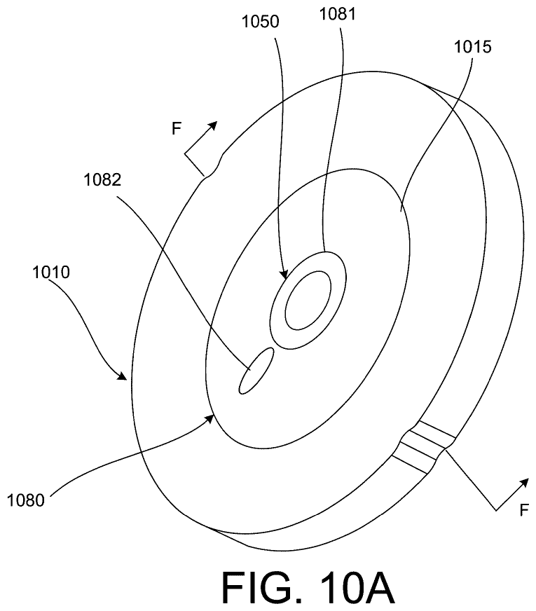

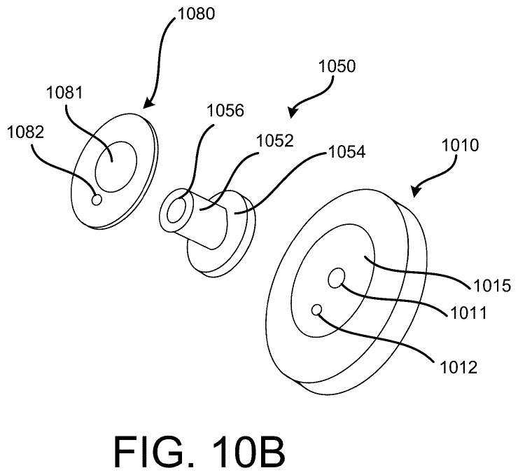

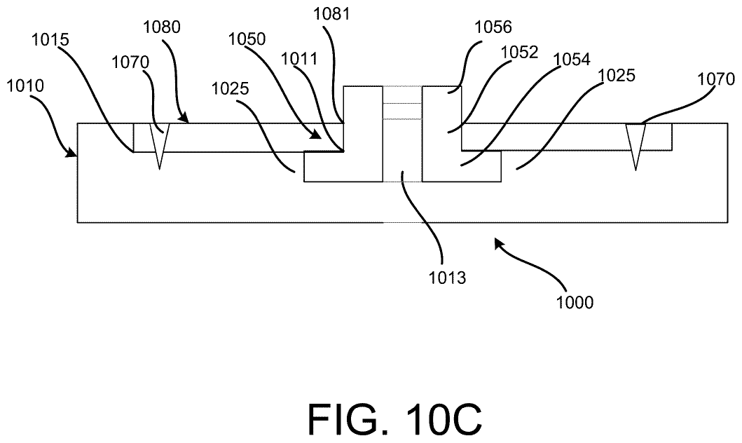

FIGS. 10A-10C illustrate a retention mechanism 1000 provided through the mechanical coupling of a seal to a base plate. In particular, FIG. 10A is a perspective view of a base plate 1010 and seal 1050. FIG. 10B is an exploded perspective view of the base plate 1010 and seal 1050 shown in FIG. 10A. FIG. 10C is a cross-sectional view, taken along line F-F of FIG. 10A, illustrating the retention mechanism 1000 provided by the mechanical coupling of the seal 1050 and the base plate 1010.

The base plate 1010 includes a first opening 1011 and a second opening 1012, similar to the first opening 411 and the second opening 412 in the base plate 410, connecting the chamber 480 to the first fluid passageway 413 of the example valve device 400. In the example shown in FIGS. 10A-10C, the seal retention mechanism 1000 is provided by the positive retention of the seal 1050 relative to the base plate 1010 due to the attachment of a support plate 1080 coupled to the base plate 1010, with the seal 1050 positioned therebetween.

In the example arrangement shown in FIGS. 10A-10C, the seal 1050 includes a body portion 1052, with a flange portion 1054 extending outward, for example radially outward, from a first end portion of the body portion 1052, and a sealing portion 1056 provided at a second end portion of the body portion 1052. In the example shown in FIGS. 10A-10C, the flange portion 1054 of the seal 1050 is positioned in an undercut portion 1025 of the base plate 1010, with the body portion 1052 of the seal 1050 extending upward (in the example orientation shown in FIG. 10C) and positioned in the first opening 1011 and extending through the first opening 1081 in the support plate 1080. In this example arrangement, the body portion 1052 of the seal extends along an inner portion of the first fluid passageway 1013. The support plate 1080 is coupled in a recessed portion 1015 formed in an upper portion of the base plate 1010 (in the example orientation shown in FIG. 10C), with the seal 1050 positioned therebetween, to provide for positive retention of the seal 1050 relative to the base plate 1010. In some examples, the support plate 1080 includes a first opening 1081 corresponding to the first opening 1011 in the base plate 1010, and to the first fluid passageway 1013 through the body portion 1052 of the seal 1050. In some examples, the support plate 1080 includes a second opening 1082 corresponding to a second opening 1012 in the base plate 1010. In the example shown in FIGS. 10A-10C, the support plate 1080 is coupled to the base plate 1010 by at least one weld 1070, simply for purposes of discussion and illustration. Other coupling devices may be used to couple the support plate 1080 to the base plate 1010.

In the example arrangement shown in FIGS. 10A-10C, the flange portion 1054 of the seal 1050 provides a seating surface of the seal 1050 that mates with, or engages, an undercut portion 1025 formed in the base plate 1010. This interface, between the flange portion 1054 of the seal 1050 and the undercut portion 1025 of the base plate 1010, together with the coupling of the support plate 1080 to the recessed portion 1015 formed at the upper portion of the base plate 1010 and an upper portion of the flange portion 1054 of the seal 1050, forms the retention mechanism 1000 that positively retains the position of the seal 1050 relative to the base plate 1010.

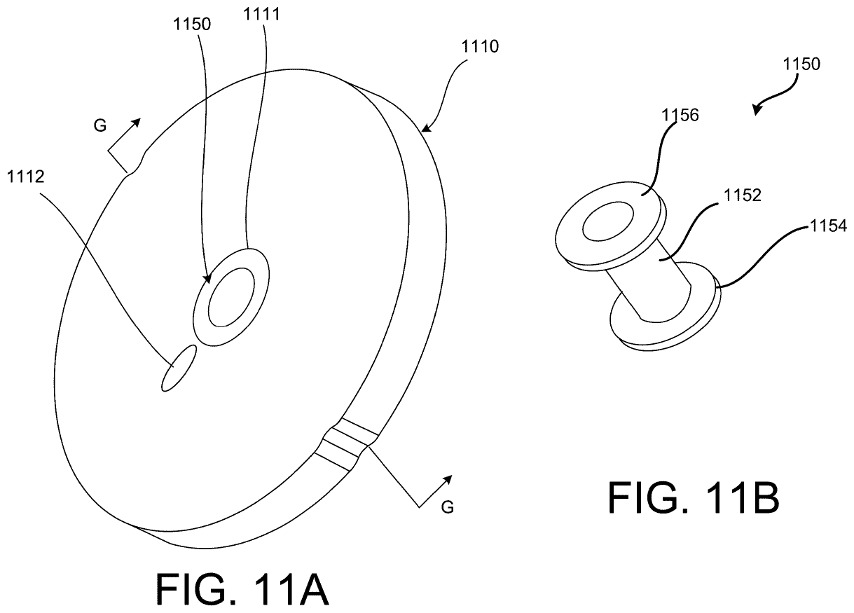

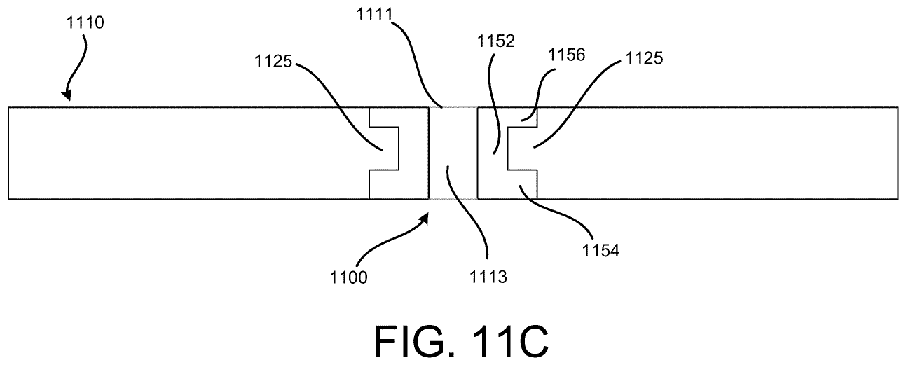

FIGS. 11A-11C illustrate a retention mechanism 1100 provided through press fit or interference fit of a seal to a base plate. In particular, FIG. 11A is a perspective view of a base plate 1110 and scal 1150. FIG. 11B is a perspective view of the seal 1150 shown in FIG. 11A. FIG. 11C is a cross-sectional view, taken along line G-G of FIG. 11A, illustrating the retention mechanism 1100 provided by the press fit, or interference fit, of the seal 1150 and the base plate 1110.

The base plate 1110 includes a first opening 1111 and a second opening 1112, similar to the first opening 411 and the second opening 412 in the base plate 410, connecting the chamber 480 to the first fluid passageway 413 of the example valve device 400. In the example shown in FIGS. 11A-11C, the seal retention mechanism 1100 is provided by the positive retention of the seal 1150 relative to the base plate 1110 due to the press fit, or interference fit, of the seal 1150 in the base plate 1110, as well as the interaction of flange portions of the seal with opposite side surfaces of the base plate 1110.

In the example arrangement shown in FIGS. 11A-11C, the seal 1150 includes a body portion 1052, with a first flange portion 1154 extending outward, for example radially outward, from a first end portion of the body portion 1152, and a second flange portion 1156 extending outward, for example, radially outward, from a second end portion of the body portion 1152. At least one of the first flange portion 1154 or the second flange portion 1156 of the seal 1150 may define a sealing portion of the seal 1150. In the example shown in FIGS. 11A-11C, an undercut portion 1125 of the base plate 1110 is positioned between the first flange portion 1154 and the second flange portion 1156 of the base plate 1110, fitted against the body portion 1152 of the seal 1150. In this example arrangement the body portion 1152 of the seal 1150 extending is positioned in the first opening 1111 and extends along an inner portion of the first fluid passageway 1113.

In some examples, the seal 1150 is a pre-molded insert that is made of an elastomer material that can be deformed for insertion into the first opening 1111 in the base plate 1110. In some examples, the seal 1150 is over-molded into the base plate 1110. In this example arrangement, the interaction between the first flange portion 1154 and a corresponding recessed area defined by the undercut portion 1125 of the base plate 1110, together with the interaction between the second flange portion 1156 and a corresponding recessed area defined by the undercut portion 1125 of the base plate 1110, defines the retention mechanism 1100 that positively retains the seal 1150 in the base plate 1110.

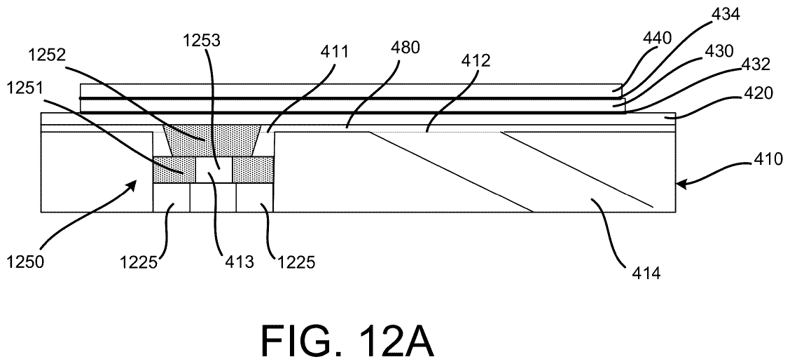

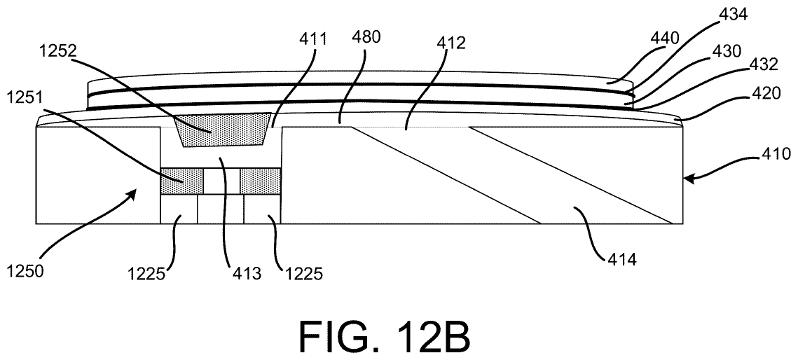

FIGS. 12A and 12B are cross-sectional views, illustrating the incorporation of an example seal 1250 into the example valve device 400 described above. The example valve device 400 is shown in FIGS. 12A and 12B, simply for purposes of discussion and illustration. The principles to be described herein can be applied to other types of fluidics components such as, for example pump devices, combined pump and valve devices, and the like, and other such components which would benefit from the retention of a sealing device during a fluid flow operation.

FIG. 12A illustrates the example valve device 400 in the closed position. FIG. 12B illustrates the example valve device 400 in the open position. A seal 1250 is installed in the first fluid passageway 413, formed in communication with the fluid chamber 480 of the valve device 400 via the first opening 411 in the base plate 410. The seal 1250 includes a first seal portion 1251 mounted on a stepped portion 1225 of the base plate 410. The stepped portion 1225 extends inward, for example radially inward, from a wall portion of the first fluid passageway toward a central portion of the first fluid passageway. The first seal portion 1251 can be fixed to the stepped portion 1225 of the base plate 410 in any number of ways including, for example, an adhesive, an epoxy, or other such bonding agent. The seal 1250 includes a second seal portion 1252 fixed to an interior facing side of the diaphragm 420, at a position corresponding to the first seal portion 1251. The second seal portion 1251 can be fixed to the diaphragm 420 in any number of ways including, for example, an adhesive, an epoxy, or other such bonding agent. The second seal portion 1252 is configured to selectively contact the first seal portion 1251 so as to selective open and close the first fluid passageway 413.

In the example arrangement shown in FIGS. 12A and 12B, the first seal portion 1251 includes an opening 1253. The opening 1253 in the first seal portion 1251 is aligned with an opening defined by the stepped portion 1225 of the base plate 410, and the first fluid passageway 413. In the closed position shown in FIG. 12A, the piezoelectric element 440 (and the isolation layer 430 and the diaphragm 420 coupled thereto) is not deformed or deflected. In the undeformed, or undeflected state of the piezoelectric element 440, the second seal portion 1252 extends down, through the first opening 411 and into the first fluid passageway 413, where the second seal portion 1252 is positioned against the first seal portion 1251, closing off the opening 1253 in the first seal portion 1251. Closing of the opening 1253 in the first seal portion 1251 in this manner obstructs the flow of fluid between the first fluid passageway 413 and the fluid chamber 480, thus closing the example valve device 400.

The deflected, or deformed state of the piezoelectric element 440 (and the isolation layer 430 and the diaphragm 420 coupled thereto) is shown in FIG. 12B. In the deflected, or deformed state of the piezoelectric element 440, the second seal portion 1252 is moved, together with the diaphragm 420 to which it is coupled, away from the first seal portion 1251. Thus, in the deflected or deformed state of the piezoelectric element, the second seal portion 1252 is spaced apart from the first seal portion 1251. Movement of the second seal portion 1252 away from the first seal portion 1251 in this manner opens the opening 1253 in the first seal portion 1251, thus opening the example valve device 400 and allowing fluid to flow between the first fluid passageway 413 and the fluid chamber 480. In the example arrangement shown in FIGS. 12A and 12B, the example seal 1250 is positively retained as fluid flows through the fluid chamber 480 due to the coupling of the first seal portion 1251 to the stepped portion 1225 of the base plate 410, and the coupling of the second seal portion 1252 to the diaphragm 420.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An implantable fluid operated inflatable device, comprising:
a fluid control system configured to control fluid flow between a fluid reservoir and an inflatable member, the fluid control system including:
at least one fluid passageway defined within a housing; and
at least one fluidic component positioned in the at least one fluid passageway and configured to control a flow of fluid through the at least one fluid passageway, the at least one fluidic component including:
a fluid chamber;
a seal positioned between the at least one fluid passageway and the fluid chamber, and configured to seal an interface between the at least one fluid passageway and the fluid chamber in a closed position of the at least one fluidic component; and
a seal retention mechanism configured to retain a position of the seal in an open position of the at least one fluidic component.

2. The implantable fluid operated inflatable device of claim 1, wherein the at least one fluidic component includes a valve device, the valve device including:
a base plate;
an opening formed in the base plate, connecting the fluid chamber to the at least one fluid passageway; a diaphragm coupled to the base plate, wherein the fluid chamber is defined between the base plate and the diaphragm; and
a piezoelectric element coupled to the diaphragm, wherein:
in response to a first voltage applied to the piezoelectric element, the diaphragm is in a closed position relative to the base plate, with the seal sealing an interface between the at least one fluid passageway and the fluid chamber, and
in response to a second voltage applied to the piezoelectric element, the diaphragm is deformed to an open position relative to the base plate to open the interface between the at least one fluid passageway and the fluid chamber, and the seal retention mechanism retains a position of the seal relative to the base plate.

3. The implantable fluid operated inflatable device of claim 2, wherein
the seal includes an O-ring positioned in a recess surrounding the opening formed in the base plate; and
the seal retention mechanism includes:
a texturing layer formed on a surface of the recess; and
a bonding layer between the texturing layer and the O-ring, the bonding layer coupling the O-ring in the recess via the texturing layer.

4. The implantable fluid operated inflatable device of claim 2, wherein
the seal includes an O-ring positioned in a recess surrounding the opening formed in the base plate; and
the seal retention mechanism includes an undercut portion formed at an opening into the recess, wherein a dimension of the opening is less than a corresponding dimension of the O-ring such that the O-ring is retained in the recess by the undercut portion.

5. The implantable fluid operated inflatable device of claim 2, wherein the seal is an elastomer O-ring, and wherein the seal retention mechanism includes a recess surrounding the opening formed in the base plate, with the O-ring received in the recess, wherein
a dimension of an inner peripheral portion of the recess is greater than a corresponding dimension of the O-ring, such that the O-ring is retained in the recess by a compression force exerted by the O-ring on the inner peripheral portion of the recess, or
a dimension of an outer peripheral portion of the recess is less than a corresponding dimension of the O-ring, such that the O-ring is retained in the recess by a tensile force exerted by the O-ring on the outer peripheral portion of the recess.

6. The implantable fluid operated inflatable device of claim 2, wherein
the seal is insert-molded into the base plate, the seal including:
a body portion;
a flange portion at a first end portion of the body portion;
a sealing portion at a second end portion of the body portion; and
an opening formed in the body portion, and
the seal retention mechanism includes:
an undercut portion formed in a body portion of the base plate and configured to engage the flange portion of the seal; and
molding material received through the opening in the body portion of the seal and integrally formed between a body portion and a central opening of the base plate.

7. The implantable fluid operated inflatable device of claim 2, wherein
the seal includes:
a body portion;
a flange portion at a first end portion of the body portion;
a sealing portion at a second end portion of the body portion, the sealing portion extending through a corresponding opening in the base plate; and
an opening extending through the body portion, from the first end portion to the second end portion of the body portion, and
the seal retention mechanism includes:
an undercut portion formed in a body portion of the base plate and configured to engage an upper portion of the flange portion of the seal; and
a support plate coupled to a bottom portion of the base plate and configured to engage a bottom portion of the flange portion of the seal, such that the flange portion of the seal is retained between the base plate and the support plate.

8. The implantable fluid operated inflatable device of claim 2, wherein
the seal includes:
a body portion;
a flange portion at a first end portion of the body portion;
a sealing portion at a second end portion of the body portion; and
an opening extending through the body portion, from the first end portion to the second end portion of the body portion, and
the seal retention mechanism includes:
a recessed portion formed in an upper portion of the base plate and configured to receive a lower portion of the flange portion of the seal; and
a support plate coupled to the upper portion of the base plate and configured to engage an upper portion of the flange portion of the seal, such that the flange portion of the seal is retained between the support plate and the base plate, with the sealing portion of the seal extending through an opening in the support plate.

9. The implantable fluid operated inflatable device of claim 2, wherein
the seal includes:
a body portion;
a first flange portion at a first end portion of the body portion;
a second flange portion at a second end portion of the body portion, the second flange portion defining a sealing portion of the seal; and
an opening extending through the first flange portion, the body portion, and the second flange portion, and
the seal retention mechanism includes an undercut portion of the body portion of the base plate positioned against an outer peripheral portion of the body portion of the seal, and engaging the first flange portion and the second flange portion.

10. The implantable fluid operated inflatable device of claim 2, wherein the seal includes:
a first seal portion fixed on a stepped portion of the base plate, the stepped portion extending into the at least one fluid passageway from a body portion of the base plate; and
a second seal portion fixed to the diaphragm, wherein
in the closed position of the diaphragm relative to the base plate, the second seal portion is positioned against the first seal portion of the seal to block an opening formed in the first seal portion and close the at least one fluid passageway; and
in the open position of the diaphragm relative to the base plate, the second seal portion is spaced apart from the first seal portion of the seal, so as to open the opening formed in the first seal portion and open the at least one fluid passageway.

11. A fluid control system for an implantable fluid operated inflatable device, comprising:
at least one fluid passageway defined within a housing; and
a fluid control device positioned in the at least one fluid passageway, the fluid control device including:
a base plate;
a diaphragm coupled to the base plate;
a fluid chamber defined between the base plate and the diaphragm;
an opening formed in the base plate, the opening connecting the fluid chamber to a fluid passageway of the fluid control device;
a seal provided at the opening and configured to form a seal between the fluid passageway and the fluid chamber in a closed state of the fluid control device; and
a seal retention mechanism engaged with the seal and configured to retain a position of the seal in an open state of the fluid control device.

12. The fluid control system of claim 11, wherein
the seal includes an O-ring positioned in a recess surrounding the opening formed in the base plate; and
the seal retention mechanism includes:
a texturing layer formed on a surface of the recess; and
a bonding layer between the texturing layer and the O-ring, the bonding layer coupling the O-ring in the recess via the texturing layer.

13. The fluid control system of claim 11, wherein
the seal includes an elastomer O-ring received in a recess surrounding the opening formed in the base plate; and
the seal retention mechanism includes an undercut portion formed at an opening into the recess, wherein a dimension of the opening is less than a corresponding dimension of the elastomer O-ring such that the elastomer O-ring is retained in the recess by the undercut portion.

14. The fluid control system of claim 11, wherein the seal is an elastomer O-ring, and wherein the seal retention mechanism includes a recess surrounding the opening formed in the base plate, with the elastomer O-ring received in the recess, wherein
a dimension of an inner peripheral portion of the recess is greater than a corresponding dimension of the O-ring, such that the O-ring is retained in the recess by a compression force exerted by the O-ring on the inner peripheral portion of the recess, or
a dimension of an outer peripheral portion of the recess is less than a corresponding dimension of the O-ring, such that the O-ring is retained in the recess by a tensile force exerted by the O-ring on the outer peripheral portion of the recess.

15. The fluid control system of claim 11, wherein
the seal is insert-molded into the base plate, the seal including:
a body portion;
a flange portion at a first end portion of the body portion;
a sealing portion at a second end portion of the body portion; and
an opening formed in the body portion, and
the seal retention mechanism includes:
an undercut portion formed in a body portion of the base plate and configured to engage the flange portion of the seal; and
molding material received through the opening in the body portion of the seal and integrally formed between a body portion and a central opening of the base plate.

16. The fluid control system of claim 11, wherein
the seal includes:
a body portion;
a flange portion at a first end portion of the body portion;
a sealing portion at a second end portion of the body portion, the sealing portion extending through a corresponding opening in the base plate; and
an opening extending through the body portion, from the first end portion to the second end portion of the body portion, and
the seal retention mechanism includes:
an undercut portion formed in a body portion of the base plate and configured to engage an upper portion of the flange portion of the seal; and
a support plate coupled to a bottom portion of the base plate and configured to engage a bottom portion of the flange portion of the seal, such that the flange portion of the seal is retained between the base plate and the support plate.

17. The fluid control system of claim 11, wherein
the seal includes:
a body portion;
a flange portion at a first end portion of the body portion;
a sealing portion at a second end portion of the body portion; and
an opening extending through the body portion, from the first end portion to the second end portion of the body portion, and
the seal retention mechanism includes:
a recessed portion formed in an upper portion of the base plate and configured to receive a lower portion of the flange portion of the seal; and
a support plate coupled to the upper portion of the base plate and configured to engage an upper portion of the flange portion of the seal, such that the flange portion of the seal is retained between the support plate and the base plate, with the sealing portion of the seal extending through an opening in the support plate.

18. The fluid control system of claim 11, wherein
the seal includes:
a body portion;
a first flange portion at a first end portion of the body portion;
a second flange portion at a second end portion of the body portion, the second flange portion defining a sealing portion of the seal; and
an opening extending through the first flange portion, the body portion, and the second flange portion, and
the seal retention mechanism includes an undercut portion of the body portion of the base plate positioned against an outer peripheral portion of the body portion of the seal, and engaging the first flange portion and the second flange portion.

19. The fluid control system of claim 11, wherein the seal includes:
a first seal portion fixed on a stepped portion of the base plate, the stepped portion extending into the at least one fluid passageway from a body portion of the base plate; and
a second seal portion fixed to the diaphragm, wherein
in the closed state of the diaphragm relative to the base plate, the second seal portion is positioned against the first seal portion of the seal to block an opening formed in the first seal portion and close the at least one fluid passageway; and
in the open state of the diaphragm relative to the base plate, the second seal portion is spaced apart from the first seal portion of the seal, so as to open the opening formed in the first seal portion and open the at least one fluid passageway.

20. The fluid control system of claim 11, wherein the fluid control device is one of a valve device, a pump device, or a combined pump and valve device.
